(19) United States
(12) Patent Application Publication
(10) Pub. No.: US 2013/0178697 A1
(43) Pub. Date: Jul. 11, 2013

(54) SMOOTH SURFACE GASTRIC BAND DEVICE AND SYSTEM

(71) Applicant: Julian Omidi, Beverly Hills, CA (US)

(72) Inventors: Julian Omidi, Beverly Hills, CA (US); Kaveh Khast, Beverly Hills, CA (US)

(73) Assignee: Julian Omidi, Beverly Hills, CA (US)

(21) Appl. No.: 13/722,350

(22) Filed: Dec. 20, 2012

Related U.S. Application Data
(60) Provisional application No. 61/582,344, filed on Dec. 31, 2011.

(51) Int. Cl. A61F 5/00

(52) U.S. Cl. CPC A61F 5/005
USPC 600/37

(57) ABSTRACT

A gastric band that includes a band portion having securing system that secures the gastric band in a closed position, and an inflatable portion associated with the band portion that includes a stomach contacting wall. The tissue contacting wall includes an exterior surface and an interior surface that includes at least one relief area that defines a pillow portion in the tissue contacting wall.

This application claims the benefit of U.S. Provisional Application No. 61/582,344, filed Dec. 31, 2011, which is herein incorporated by reference in its entirety.

### FIELD OF THE INVENTION

The present invention relates to a gastric band, and more specifically to a gastric band with a smooth interior surface.

### BACKGROUND OF THE INVENTION

Gastric bands are used to aid patients in the loss of weight to combat obesity. Examples of gastric bands can be found in U.S. Pat. No. 7,811,298 to Birk and U.S. Pat. No. 5,226,429 to Kuzmak, the entireties of which are hereby incorporated by reference. In use, prior art gastric bands can irritate tissues by the undulating topography of the surfaces next to the tissue during normal movements such as peristaltic contractions of the esophagus.

### SUMMARY OF THE PREFERRED EMBODIMENTS

In accordance with a first aspect of the present invention there is provided a gastric band that includes a band portion having a securing system that secures the gastric band in a closed position, and an inflatable portion associated with the band portion that includes a stomach contacting wall. The tissue contacting wall includes an exterior surface and an interior surface that includes at least one relief area that defines a pillow portion in the tissue contacting wall. In a preferred embodiment, the interior surface of the tissue contacting wall includes a series of relief areas that define a series of pillow portions. The inflatable portion has a deflated state and an inflated state, and the pillow portions extend inwardly when the inflatable portion is in the inflated state.

In accordance with another aspect of the present invention there is provided a gastric band that is movable between a closed position and an open position and that includes a band portion that has a securing system that secures the gastric band in the closed position, and an inflatable portion associated with the band portion that is operable between a deflated state and an inflated state. The inflatable portion includes a series of pillow portions. When the gastric band is in the closed position and the inflatable portion is in a deflated state, the pillow portions generally define a first inner diameter. When the gastric band is in the closed position and the inflatable portion is in an inflated state, the pillow portions generally define a second inner diameter. Preferably, the first diameter is larger than the second diameter. In a preferred embodiment, the pillow portions are separated by a series of intermediate members, and when the gastric band is in the closed position and the inflatable portion is in an inflated state, the intermediate members define a third diameter. The third diameter is larger than the second diameter. When the gastric band is in the closed position and the inflatable portion is in the inflated state the third diameter is approximately the same as the first diameter.

In accordance with another aspect of the present invention there is provided a method that includes the steps of obtaining a gastric band that includes an inflatable portion that is operable between a deflated state and an inflated state, and inflating the inflatable portion such that it is in the inflated state. The gastric band is initially in the deflated state and includes a tissue contacting wall that has an interior surface and an exterior surface. The exterior surface is generally flat when the inflatable portion is in the deflated state and the exterior surface of the tissue contacting wall includes a series of bulging pillow portions when the inflatable portion is in the inflated state. In a preferred embodiment, the tissue contacting wall includes a series of relief areas that each defines one of the pillow portions. The tissue contacting wall preferably includes a plurality of intermediate members. Each intermediate member is disposed between two pillow portions. The intermediate members have a greater thickness than the pillow portions.

The invention, together with additional features and advantages thereof, may be best understood by reference to the following description.

### BRIEF DESCRIPTION OF THE DRAWINGS

FIG. 1 is a top elevational view of a gastric band in an open position in accordance with a preferred embodiment of the present invention;

FIG. 2 is a top elevational v of the gastric band of FIG. 1 in an inflated state;

FIG. 3 is a bottom elevational view of the gastric band of FIG. 1 in a closed position and a deflated state;

FIG. 4 is a bottom elevational view of the gastric band of FIG. 1 in a closed position and an inflated state;

FIG. 5 is a cross-sectional view of the gastric band of FIG. 1;

FIG. 6 is a cross-sectional view of the gastric band of FIG. 1 in an inflated state;

FIG. 7 is a detailed cross-sectional view of a portion of the gastric band of FIG. 1; and

FIG. 8 is another cross-sectional view of the gastric band of FIG. 1.

### DETAILED DESCRIPTION OF THE PREFERRED EMBODIMENTS

Selected embodiments will now be explained with reference to the drawings. It will be apparent to those skilled in the art from this disclosure that the following descriptions of the embodiments are provided for illustration only and not for the purpose of limiting the invention as defined by the appended claims and their equivalents.

Referring to FIGS. 1-8, a gastric band 10 is illustrated in accordance with an embodiment of the present invention. It should be noted that the descriptions that follow, for example, terms of the gastric band device and system is described for illustrative purposes and the underlying system can apply to any number and multiple types of obesity and weight control treatments. Generally, the gastric band 10 includes a band portion 12, an inflatable portion 16, a fluid inlet fitting 18 and a fill tube 14 that includes an interior in fluid communication with the inflatable portion 16.

In one embodiment, the smooth surfaced gastric band 10 and system can be configured using hypoallergenic materials. The smooth surfaced gastric band 10 and system ea be configured to include metal or ceramic injection stops and can be configured to include saline solutions for uses such as inflating the smooth surfaced gastric band 10 using the present invention.

In a preferred embodiment, the gastric band 10 is made of a silicon material that is hypoallergenic. However, this is not a limitation on the presentation, and the gastric band 10 or different components thereof can be made of other materials. As is known in the art, the gastric band 10 includes the fluid inlet fitting 18. Any type of fluid inlet fitting 18 is within the scope of the present invention. In a exemplary embodiment, the inflation fluid is a saline solution that is introduced through the fluid inlet fitting 18, which can be fitted inside and against the abdominal muscle, or elsewhere. Saline solution can be injected through the fluid inlet fitting 18. In a preferred embodiment, the fluid inlet fitting 18 is a self-healing silicon with a backstop to prevent the, for example, syringe needle from being inserted and passing through the inlet and injecting saline directly into the body. However, this is not a limitation on the present invention. The backstop can be made of materials such as titanium, ceramic and other hard material that is hypoallergenic.

As shown in FIGS. 1-4, in a preferred embodiment, the inflatable portion 16 extends inwardly from the Ix id portion 12 and includes an upper wall 20, lower wall 22, tissue contacting wall 24, and a pair of end walls 26. In other words, in a preferred embodiment, the band portion 12 and the inflatable portion 16 form a unitary component. In another embodiment, the inflatable portion and band portion can be separate components. For example, the inflatable portion can extend around the band portion.

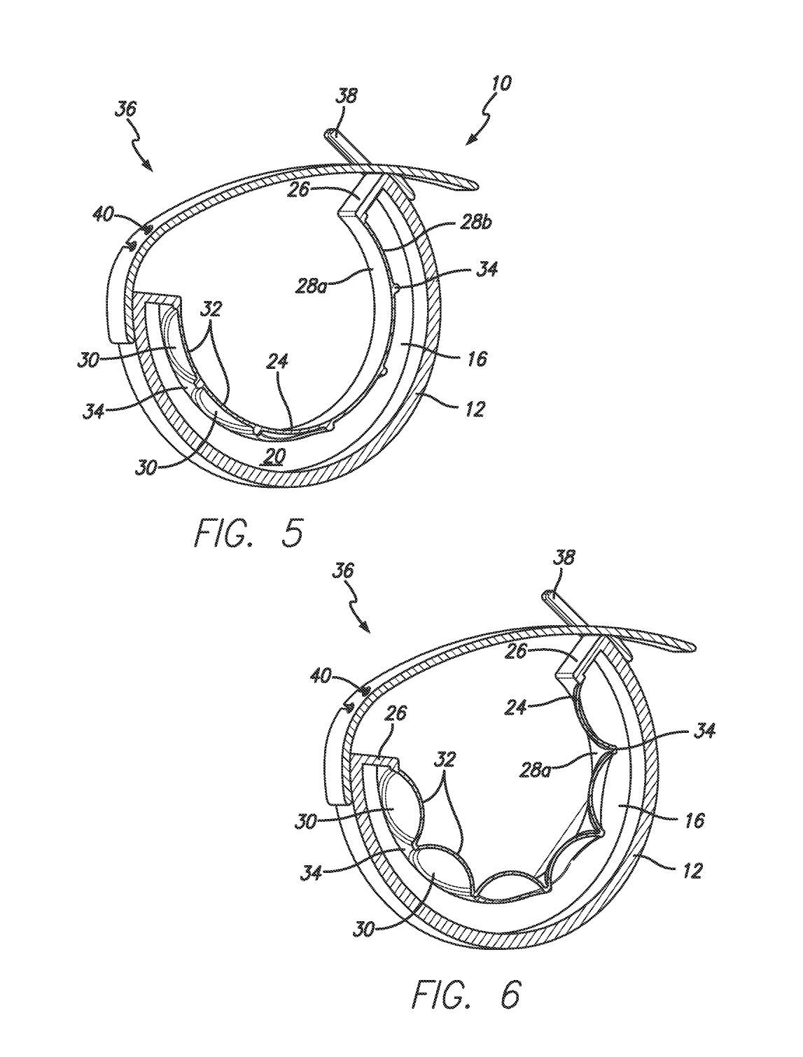

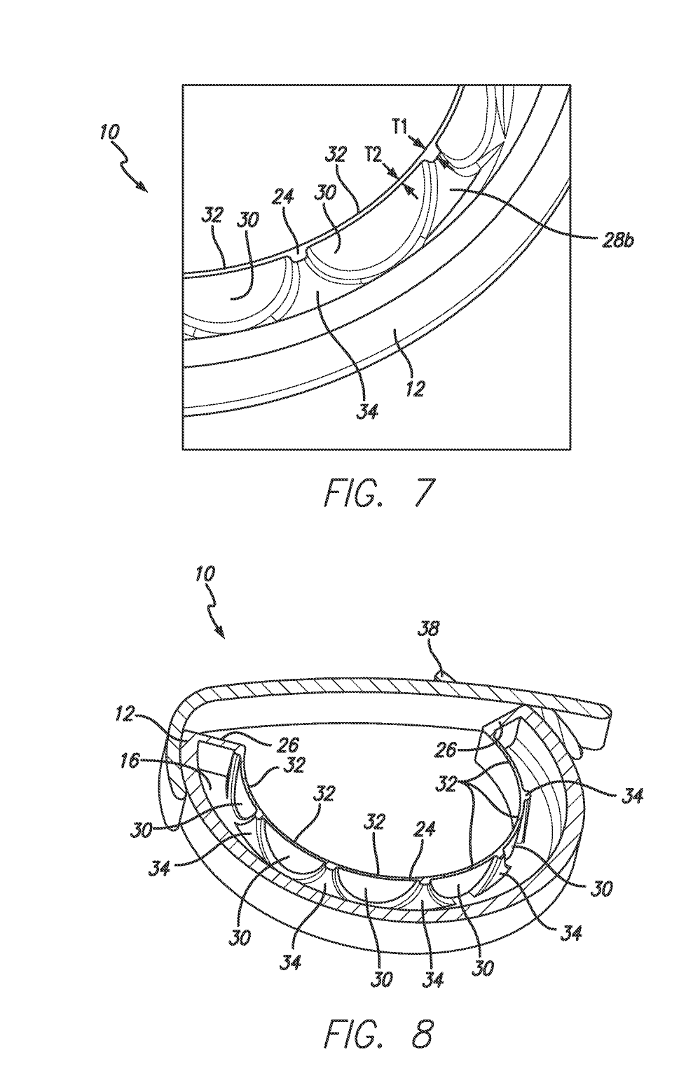

As shown in FIGS. 5-8, the tissue contacting wall 24 includes an interior surface 28a and an exterior surface 28b. In a preferred embodiment, the tissue contacting wall 24 includes relief area, 30 defined in the interior surface 28a that each define a pillow portion 32. As a result of the definition of the relief areas 30 in the tissue contacting wall 24, intermediate members 34 that are disposed between relief areas 30 and pillow portions 32 are formed. It will be understood that in the deflated state, the tissue contacting wall 24 (at the intermediate portions 34) has a first thickness T1 and the pillow portions 32 have a second thickness T2, as shown in FIG. 7.

Preferably, the relief areas 30 and respective pillow portions 32 are generally circular in shape. However, this is not a limitation and any shape is within the scope of the present invention. For example, in another embodiment, the relief areas 30 and respective pillow portions 32 can be square, rectangular, ovular or any other shape. In another embodiment, the relief areas 30 can be defined in the exterior surface 28b of the tissue contacting wall 24.

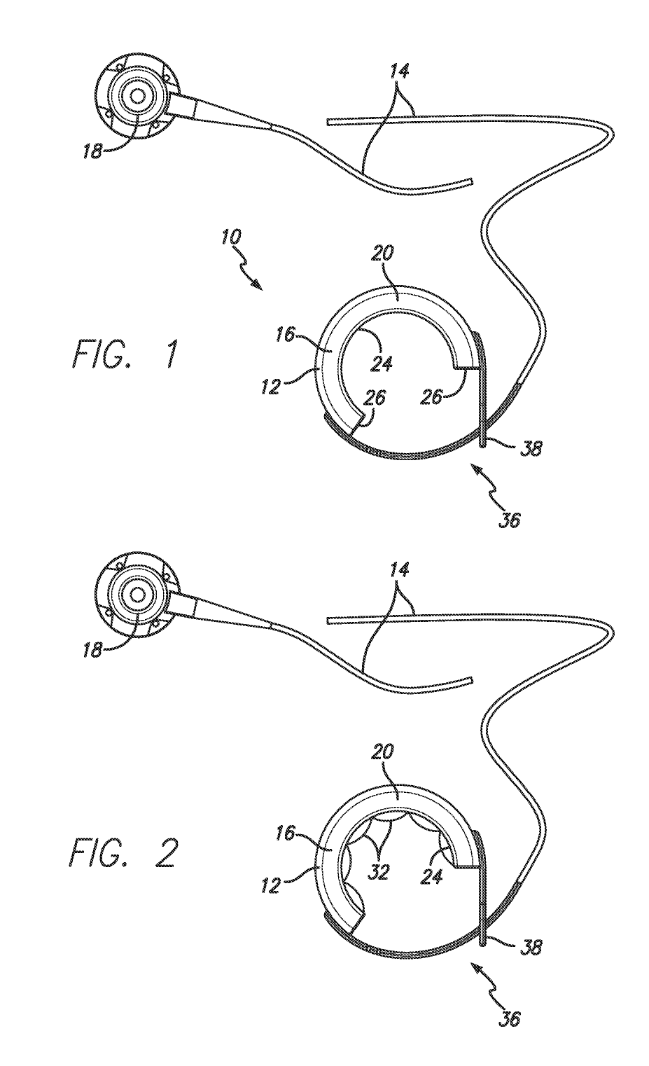

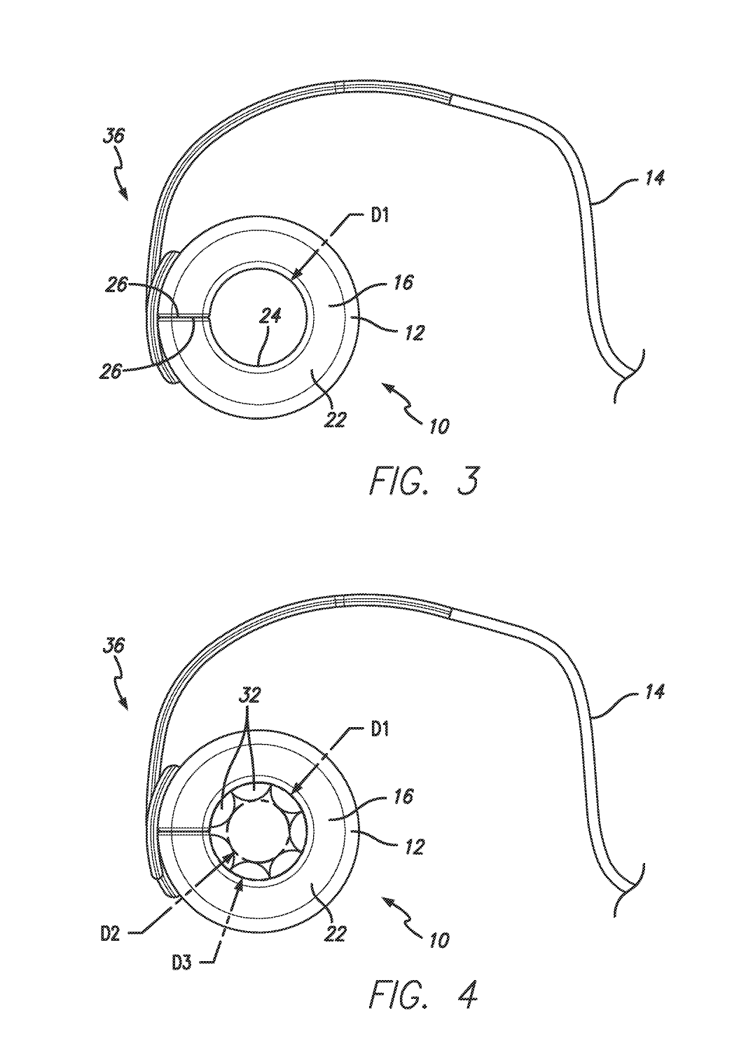

The gastric band 10 is movable between an open state (see FIGS. 1-2) and a closed state (see FIGS. 3-4), by way of a securing system 36. The securing system 36 shown in the figures includes tabs 38 for retaining the band portion 12 and openings 40 therein through which a suture can be passed for securing the gastric band 10 in the closed state. However, this type of securing system is not a limitation on the present invention and any type of securing system is within the scope of the present invention.

The inflatable portion 16 is operable between a deflated or normal state (see FIGS. 1, 3 and 5) and an inflated state (see FIGS. 2, 4 and 6). In the deflated state, the interior surface 28a of the tissue contacting wall 24 has a relatively flat and smooth shape, as show FIG. 3, and, in the closed position, a relatively constant diameter with no undulating surfaces. In the inflated state, the pillow portions 32 extend inwardly, thus causing the interior surface 28a of the tissue contacting wall 24 to have the shape shown in FIG. 4. The inflated state is attained when saline or other fluid is introduced into the inflatable portion to at least a predetermined pressure to cause the pillow portions 32 to bulge, flex or otherwise extend inwardly, as is described below.

In use, the gastric band 10 is applied around the esophagus just above the entrance to the stomach. As the saline fluid is injected, the interior pressure increases. The components of the band portion 12 and inflatable portion 16 other than the pillow portions 32 (e.g., upper wall 20, lower wall 22, end walls 26, and intermediate members 34) have sufficient wall thickness to not flex any significant amount. As a result, the thin walled relief areas 30 (and thus the pillow portions 32) are forced to flex outwardly by the pressure of the injected saline. The outward flexing creates a bulge, as is shown in FIGS. 2 and 4. The bulging pillow portions 32 pushes against the outer surface of the esophagus constricting the esophagus cavity. It will be appreciated that some internal pressures may cause some components of the band portion 12 and inflatable portion 16 (e.g., upper wall 20, lower wall 22, end walls 26, and intermediate members 34) to bulge or move, but that, even if this occurs, the relief areas 30 (and thus the pillow portions 32) will bulge or move more than the other components.

The constricted esophagus cavity presents the patient with a sensation of being full thus reducing the total amount of food ingested at any meal. As the patient begins weight loss the volume of saline fluid can be reduced. The saline is extracted through the inlet fitting using a syringe to withdraw a portion of the saline fluid. The reduced volume lessens the internal pressure of the gastric band 10. The reduction in the internal pressure decreases the degree of pillow portion 32 flexing and the depth of the bulge. This decreases the constriction of the esophagus and allows the patient to increase food intake by a prescribed measure.

As shown in FIG. 3, in the closed position and with the inflatable portion 16 in the deflated state, the gastric band 10 has a generally circular shape and the interior surface defines an inner diameter D1. It is understood that in practical use, the gastric band 10 may not be exactly circular, but that it may be somewhat offset or lopsided. This is not a limitation on the present invention. As shown in FIG. 4, in the closed position and with the inflatable portion 16 in the inflated state, the bulged pillow portions 32 define a second diameter D2 and the interior surface of the intermediate members 34 define a third diameter D3. The second diameter D2 is smaller than both the first diameter D1 and the third diameter D3. And, in an embodiment where the intermediate members 34 do not flex, the third diameter D3 and the first diameter DI are about the same. In an embodiment where the intermediate members 34 do flex, the third diameter D3 is smaller than the first diameter D1, but the second diameter D2 is still smaller than the third diameter D3.

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having” and their derivatives. Also, the terms “part,” “section,” “portion,” “member” or “element” when used in the singular can have the dual meaning of a single part or a plurality of parts. As used herein to describe the above embodiment(s), the following directional terms “forward,” “rearward,” “rightward,” “leftward,” “outward,” “forward,” “inward,” “downward,” “upward,” “above,” “below,” “vertical,” “horizontal,” and “transverse” as well as any other similar directional terms refer to those directions of a bicycle equipped with the bicycle control device. Accordingly, these terms, as utilized to describe the bicycle control device should be interpreted relative to a bicycle equipped with the bicycle control device as used in the normal riding position on a level surface. Finally, terms of degree such as “substantially,” “about” and “approximately” as used herein mean a reasonable amount of deviation of the modified term such that the end result is not significantly changed.

While certain aspects of the disclosure are presented below in certain claim forms, the inventors contemplate the various aspects of the disclosure in any number of claim forms. Any claims intended to be treated under 35 U.S.C. §112, ¶6 will begin with the words “means for”. Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A gastric band comprising:
a band portion that includes a securing system that secures the gastric band in a closed position, and
an inflatable portion associated with the band portion and that includes a stomach contacting wall, wherein the tissue contacting wall includes an interior surface and an exterior surface, wherein the interior surface includes at least one relief area that defines a pillow portion in the tissue contacting wall.

2. The gastric band of claim 1 wherein the interior surface of the tissue contacting wall includes a series of relief areas that define a series of pillow portions.

3. The gastric band of claim 2 wherein the inflatable portion has a deflated state and an inflated state, and wherein the pillow portions extend inwardly when the inflatable portion is in the inflated state.

4. The gastric band of claim 3 wherein the relief areas are generally circular in shape.

5. The gastric band of claim 3 wherein the inflatable portion extends inwardly from the band portion.

6. The gastric band of claim 5 wherein the inflatable portion further comprises upper and lower walls and two end walls.

7. A gastric band movable between a closed position and an open position, wherein the gastric band comprising:
a band portion that includes a securing system that secures the gastric band in the closed position, and
an inflatable portion associated with the band portion that is operable between a deflated state and an inflated state, wherein the inflatable portion includes a series of pillow portions,
wherein when the gastric band is in the closed position and the inflatable portion is in a deflated state, the pillow portions generally define a first inner diameter,
wherein when the gastric band is in the closed position and the inflatable portion is in an inflated state, the pillow portions generally define a second inner diameter, and wherein the first diameter is larger than the second diameter.

8. The gastric band of claim 7 wherein the pillow portions are separated by a series of intermediate members, and wherein when the gastric band is in the closed position and the inflatable portion is in an inflated state, the intermediate members define a third diameter, wherein the third diameter is larger than the second diameter.

9. The gastric band of claim 8 wherein when the gastric band is in the closed position and the inflatable portion is in an inflated state the third diameter is approximately the same as the first diameter.

10. A method comprising the steps of:
obtaining a gastric band that includes an inflatable portion that is operable between a deflated state and an inflated state, wherein the gastric band is initially in the deflated state, wherein the gastric band includes a tissue contacting wall having an interior surface and an exterior surface, wherein the exterior surface is generally flat when the inflatable portion is in the deflated state, and
inflating the inflatable portion such that it is in the inflated state, wherein the exterior surface of the tissue contacting wall includes a series of bulging pillow portions when the inflatable portion is in the inflated state.

11. The method of claim 10 wherein the tissue contacting wall eludes a series of relief areas that each defines one of the pillow portions.

12. The method of claim 11 wherein the tissue contacting wall includes a plurality of intermediate members, wherein each intermediate member is disposed between two pillow portions, wherein the intermediate members have a greater thickness than the pillow portions.
